(19) United States
(12) Patent Application Publication
(10) Pub. No.: US 2021/0219912 A1
(43) Pub. Date: Jul. 22, 2021

(54) DEVICE WITH AT LEAST ONE ELECTRODE UNIT FOR ELECTROSTIMULATION OR DATA ACQUISITION BY DIAGNOSTIC INSTRUMENTS

(71) Applicant: Pierenkemper GmbH, Ehringshausen (DE)

(72) Inventors: Roger Pierenkemper, Marburg (DE); Jonas Peinze, Nendeln (LI)

(73) Assignee: Pierenkemper GmbH, Ehringshausen (DE)

(21) Appl. No.: 17/142,399

(22) Filed: Jan. 6, 2021

(30) Foreign Application Priority Data
Jan. 16, 2020 (EP) 20152235.6

(51) Int. Cl. A61B 5/00; B32B 5/02; B32B 25/10; B32B 27/40; B32B 27/36; B32B 27/12; B32B 27/34; B32B 25/14; B32B 25/02; A41D 1/00; A61N 1/04

(52) U.S. Cl. CPC A61B 5/6804; A41D 2400/32; B32B 25/10; B32B 5/022; B32B 27/40; B32B 5/028; B32B 27/36; B32B 27/12; B32B 27/34; B32B 25/14; B32B 25/02; A41D 1/002; A61N 1/0484; B32B 2307/202; B32B 2437/00; B32B 2457/00; B32B 2262/0276; B32B 2262/0261; B32B 2264/1051; B32B 5/026

(57) ABSTRACT

A device for electrostimulation or for data acquisition by diagnostic instruments has at least one electrode unit with the following layers: a) base layer with at least one electrode disposed in or on the base layer, and b) layer with one of the following layer structures: b1) plastic layer and layer of knitted fabric and plastic layer or b2) layer of knitted fabric and plastic layer or b3) plastic layer and layer of knitted fabric. The layers a) and b) are joined tightly with one another.

### CROSS REFERENCE TO RELATED APPLICATIONS

Applicant claims priority under 35 U.S.C. § 119 of European Application No. 20152235.6 filed Jan. 16, 2020, the disclosure of which is incorporated by reference.

### BACKGROUND OF THE INVENTION

### 1. Field of the Invention

The invention relates to a device for electrostimulation or for data acquisition by diagnostic instruments using at least one electrode unit.

### 2. Description of the Related Art

Treatment devices and treatment methods, for example for a face, belong to the prior art (DE 10 2014 108 315 A1). During these treatments, an electrostimulation is performed by means of electrodes knitted into a knitted carrier in a face mask. This face mask is made from a knitting material or a knitted fabric, for example a warp-knitted fabric. The electrodes consist of electrically conductive threads, which are knitted in directly during production of the face mask. This device belonging to the prior art has the disadvantage that the face mask must be designed completely prior to production, because the electrode is knitted into the face mask. A subsequent adaptation to various carriers with respect to the position of the electrodes is not possible.

Furthermore, a device for complex electromyostimulation belongs to the prior art (DE 20 2012 102 393 U1). This device belonging to the prior art comprises a suit, a stimulation unit, an operator-control unit as well as a communication unit connecting the operator-control unit and the stimulation unit for signal transmission. This suit also consists of a warp-knitted fabric. In this suit, the electrode unit is disposed on the suit. The electrodes consist of metallized polymer fibers. The contact electrodes may also consist of conductive polymer masses. These contact electrodes belonging to the prior art have the disadvantage that the polymer masses easily become brittle, especially during intensive use of the suit. If the polymer masses become brittle, the electrodes are no longer functional, and the suit can no longer be used for electrostimulation. In addition, the materials currently available on the market do not meet the strict cytotoxicity requirements imposed on products of this type.

### SUMMARY OF THE INVENTION

The technical problem underlying the invention consists in specifying a device for electrostimulation or for data acquisition by diagnostic instruments with at least one electrode unit, which ensures a good conductivity of the electrode material accompanied by a great durability of the electrodes.

This technical problem is solved by a device having the features according to the invention.

The device according to the invention with at least one electrode unit for electrostimulation or for data acquisition by diagnostic instruments is characterized in that the electrode unit has the following layers:

a) base layer with at least one electrode disposed in or on the base layer, and
b) layer with one of the following layer structures:

b1)—plastic layer and
layer of knitted fabric and
plastic layer

or

b2)—layer of knitted fabric and
plastic layer

or

b3)—plastic layer and
layer of knitted fabric
   and in that the layers a) and b) are joined tightly with one another.

The device according to the invention with at least one electrode unit may be used for electrostimulation. The at least one electrode of the electrode unit is energized with current and for this reason may be used for electrostimulation. It is also possible, however, to perform data acquisition with the device according to the invention with the at least one electrode unit, for example data acquisition by means of EKG (electrocardiogram), EMG (electromyography) or, for example, for measurement of bioimpedances, etc.

The device according to the invention has the following structure with the following layers:

a) base layer with at least one electrode disposed in or on the base layer, and
b) layer with one of the following layer structures:

b1)—plastic layer and
layer of knitted fabric and
plastic layer

or

b2)—layer of knitted fabric and
plastic layer

or

b3)—plastic layer and
layer of knitted fabric.

In the electrode unit according to the invention, the layers a) and b) are joined tightly with one another. In other words, the layer a) is joined tightly with the layer b1) or the layer a) with the layer b2) or the layer a) with the layer b3). Advantageously, the layers of the layer b1) or of the layer b2) or of the layer b3) are likewise joined tightly with one another.

Due to the structure according to the invention, it is ensured that the layer b) has a layer of a knitted fabric. In this way, this layer structure remains stable, even if the base layer with the further layer is changed frequently in its basic shape, for example bent, kinked, stretched, squashed or the like.

The device according to the invention may be disposed, for example, on a suit for electrostimulation. This suit advantageously contains at least one electrode unit. Depending on arrangement on the suit, the at least one electrode unit is more or less strongly twisted or stretched or squashed or stressed in other ways. Due to configuration of the electrode unit with the layer of knitted fabric with at least one plastic layer, the electrode unit remains stable and functional even over a long usage time, even when subjected to a strong mechanical stress.

According to an advantageous embodiment of the invention, the electrode is embroidered into the base layer. The embroidery has the advantage that the electrode may be disposed in the desired shape in the base layer. This embroidered electrode is very durable.

According to an advantageous embodiment, the electrode is embroidered with electrically conductive threads into the base layer. Advantageously, the electrically conductive threads are embroidered only into a surface of the base layer. Thus, they are not embroidered throughout the entire base layer. Retaining threads that are not electrically conductive may be disposed on the opposite side. In principle, however, the possibility also exists that the electrically conductive threads are disposed continuously through the entire base layer.

According to further advantageous embodiments, the at least one electrode in the base layer may also be disposed in or on the base layer by printing, sewing, gluing and/or inserting and fixing.

The electrode is advantageously disposed on only one side of the base layer. In other words, the base layer has the electrically conductive electrode on one side. On the other side, the base layer is electrically conductive not at all or to only a small extent.

According to a particularly preferred embodiment of the invention, it is provided that the at least one electrode is disposed in or on the prefabricated base layer. This embodiment has the advantage that the electrode may be embroidered in any desired form onto the prefabricated base layer of the electrode unit. The base layer is inherently produced in finished condition. Then the at least one electrode is disposed in or on this prefabricated base layer. If, for example, an adaptation of electrodes to particular body regions is necessary, this adaptation may be defined in the embroidering process.

It is not necessary to define the design of the entire electrode unit from the very beginning, as early as during production of the base layer. The final form in the base layer is defined only during the production of the electrode unit. In this way, electrode units with different designs may be produced inexpensively.

The at least one electrode is advantageously formed from at least one electrically conductive thread. Several electrodes may be produced with one thread or strand of thread. These electrodes are then connected in series. The possibility also exists of producing one or more electrodes from respectively one electrically conductive thread. In this case, one or more electrodes may be connected in parallel. The electrodes may be formed individually or together or in a manner that can be activated in groups.

According to a further advantageous embodiment of the invention, it is provided that the at least one plastic layer is designed to be electrically conductive. In this way, it is possible to achieve a current transmission via the plastic layer.

According to a further advantageous embodiment of the invention, it is provided that the at least one plastic layer is formed from a thermoplastic material and/or from thermoplastic elastomers and/or from conductive silicones.

These materials are particularly flexible and may be deformed together with the suit or a cuff or the like. Moreover, the flexibility is preserved over a very long time.

According to a further advantageous embodiment of the invention, it is provided that the at least one plastic layer contains electrically conductive particles. In this way, the at least one plastic layer is likewise electrically conductive. Due to the plastic layer, the user of the inventive device, for example the patient or an athlete, works up a sweat more rapidly. The conductivity is increased by the moisture formed from the sweat.

All plastic layers disposed in the layers b1), b2) or b3) may contain electrically conductive particles. The possibility also exists, however, that only some of the plastic layers disposed in the layers b1), b2) or b3) contain electrically conductive particles.

The electrically conductive particles are advantageously disposed homogeneously or at least approximately homogeneously in the at least one plastic layer.

According to a further advantageous embodiment of the invention, it is provided that the electrically conductive particles consist of silver and/or graphite. These particles can be easily introduced into the plastic layer, and they increase the electrical conductivity of the at least one plastic layer.

A further advantageous embodiment of the invention provides that the base layer consists of a nonwoven fabric. The possibility also exists, however, of using other materials for the base layer. Because the electrode unit is produced by a hot-pressing method, for example, the base layer should be heat-resistant. It should not be pressed together too strongly in a thermal pressing method.

A further advantageous embodiment of the device provides that the at least one plastic layer is formed as polyurethane film. Polyurethane film can be produced particularly inexpensively. Moreover, the electrically conductive particles can be disposed well and easily in the polyurethane film.

A particularly preferred embodiment of the invention provides that the at least one layer of knitted fabric contains
gauze and/or
elastic gauze and/or
grid-like material and/or
grid-like elastic material and/or is formed
from gauze and/or
from elastic gauze and/or
a grid-like material and/or
a grid-like elastic material.

In other words, a layer of gauze and/or of elastic gauze and/or a grid-like material and/or a grid-like elastic material is advantageously formed in the layers b1), b2) or b3).

The layers b1), b2) or b3) may also contain gauze and/or grid-like material, preferably elastic gauze and/or grid-like elastic material.

The gauze and/or the grid-like material are preferably elastic.

The knitted fabric, for example the gauze and/or the grid-like material, preferably elastic material, ensure that the at least one plastic layer does not become brittle or that it withstands higher mechanical loads. This layer of knitted fabric imparts a high stability to the electrode unit with simultaneous flexibility of the electrode unit, so that the electrode unit may be adapted to body parts of the patient or athlete. The knitted fabric, for example the gauze and/or the grid-like material are advantageously formed elastically in one direction but particularly advantageously in two directions, for example in x and in y direction, in order to ensure a best-possible adaptation of the electrode unit to a body part or to the body of a patient or athlete.

Advantageously, the knitted fabric, for example the gauze or the grid-like material, is formed from polyester. This material is inexpensive, durable for a long time and can be formed well as knitted fabric, for example in the form of gauze or grid-like material having the appropriate elasticity.

Advantageously, the device according to the invention is characterized in that the layers are designed as
layers welded together with one another by a thermal pressing method or
layers joined tightly by a thermal pressing method with a primer or
layers joined tightly with one another by laminating or
layers joined tightly with one another by a high-frequency welding or an ultrasonic welding.

Connecting the various layers tightly with one another by means of the aforesaid methods has the advantage that the method can be performed inexpensively and the flexibility of the electrode unit is nevertheless ensured. Moreover, tight, undetachable joints are formed between the layers.

According to a further advantageous embodiment of the invention, it is provided that the device with the base layer is disposed on a whole-body suit or partial body suit. The whole-body suit covers the central body as well as the extremities, whereas a partial body suit may be designed in the form, for example, of a tee shirt, a pullover or a kind of trousers. Due to this construction, it is possible, for example for an athlete, to equip a whole-body suit with the device according to the invention, so that electrostimulation, for example, may take place during the sporting activity.

According to another advantageous embodiment of the invention, it is provided that the device with the base layer is disposed on a cuff. This cuff may be disposed, for example, on the extremities of an athlete or of a patient, in order likewise to perform electrostimulation during a sporting activity or to perform electrostimulation for therapeutic purposes or to acquire data, for example in the form of an EKG or EMG.

According to a further advantageous embodiment of the invention, it is provided that the whole-body suit or the cuff consists of
polyester and elastane or
polyamide 6 or elastane or
polyamide 6.6 and elastane or
polyurethane and elastane.

These materials are particularly advantageous, because the whole-body suit or partial body suit or the cuff is configured flexibly in this way, in order to accommodate shearing movements, twisting movements, stretching and/or squashing movements of the whole or partial body suit or of the cuff during movement of the athlete or patient, so that the electrode unit with the at least one electrode remains constantly in body contact.

According to a further advantageous embodiment of the invention, the electrode has a resistance relative to the electrical conductivity of less than 100 Ohm per 10 centimeters of length of the electrode. This feature is advantageous, because the electrodes hereby have a large conductivity, and so effective electrostimulation may take place.

A particularly preferred embodiment of the device according to the invention with the at least one electrode unit provides that an embroidered electrode is used that is joined with the further layers in a hot-pressing method.

If only one plastic film that contains conductive particles is used, damage occurs easily due to the constant movement of the flat electrode. For this reason, it is advantageous to additionally provide, for example, a knitted fabric in the form of a gauze or of a grid-like elastic material.

The base layer with the electrode is likewise advantageously designed to be low-ohmic, which means that it advantageously has a high conductivity and a very low resistance.

The knitted fabric, for example the gauze, preferably elastic, or the grid-like, preferably elastic material, advantageously consists of polyester or other materials. The gauze is likewise advantageously designed to be elastic.

The at least one plastic layer consists advantageously of a PU film (polyurethane). This PU film particularly advantageously contains electrically conductive particles, for example graphite and silver. The PU film may also consist of thermoplastic material or of thermoplastic elastomers and also of conductive silicones.

If the athlete is wearing a whole-body suit or partial body suit, he/she works up a sweat during the sporting activity. In this way, the base layer, which is disposed in the direction of the skin, is permeated with sweat. Due to the increase of the moisture, the conductivity is also improved.

The plastic layers are advantageously laminated onto the knitted fabric, for example the gauze and/or the grid-like elastic material. The possibility also exists of laminating the electrode unit onto the plastic film in the thermal method.

If the device according to the invention is disposed in a whole body or partial body suit, this suit advantageously has, on the outside, a nonconductive layer, for example a layer of polyurethane. The device according to the invention with the electrode unit may have a joint to the suit, for example by means of gluing or else by means of a thermal method. Particularly advantageously, the plastic films are formed from a thin flexible material, which has a high conductivity, for example with a resistance of less than 100 ohm per 10 cm length of the electrode.

The electrically conductive fibers, which for example are embroidered, inserted or printed in, may consist, for example, of stainless steel or copper.

### BRIEF DESCRIPTION OF THE DRAWINGS

Further features and advantages of the invention are also evident on the basis of the associated drawings, in which several exemplary embodiments of the device are illustrated, without restricting the invention to these exemplary embodiments.

In the drawings, wherein similar reference characters denote similar elements throughout the several views:

FIG. 1 shows a partial body suit with electrode units;

FIG. 2 shows an electrode unit in cross section;

FIG. 3 shows a different example of an electrode unit in cross section; and

FIG. 4 shows a different example of an electrode unit in cross section.

### DETAILED DESCRIPTION OF PREFERRED EMBODIMENTS

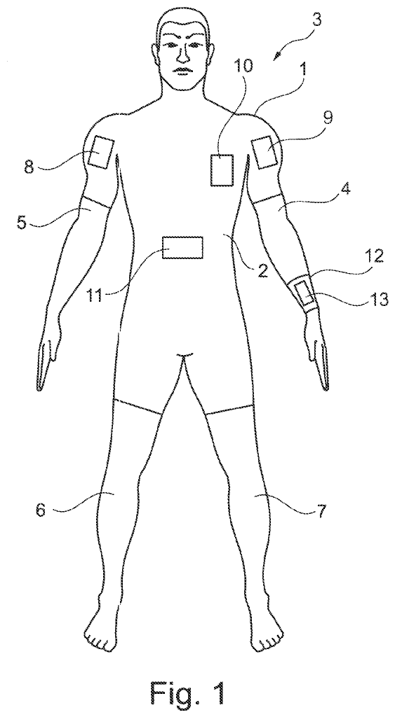

FIG. 1 shows a partial body suit 1, which covers a trunk 2 of a person 3. Extremities 4 to 7 (arms 4, 5; legs 6, 7) remain partly free of the partial body suit 1. Electrode units 8, 9, 10, 11 are disposed on the partial body suit 1. The electrode units 8, 9 are disposed in the upper arm region, the electrode 11 in the abdominal region and the electrode 10 in the heart region of the person 3. Beyond those electrodes, the person 3 wears, on the extremity 4, meaning an arm, a cuff 12, on which an electrode unit 13 is disposed. The electrode units 8, 9 and 11 may be used for electrostimulation. The electrode units 10, 13 may be used, for example, for data acquisition, for example for an EKG.

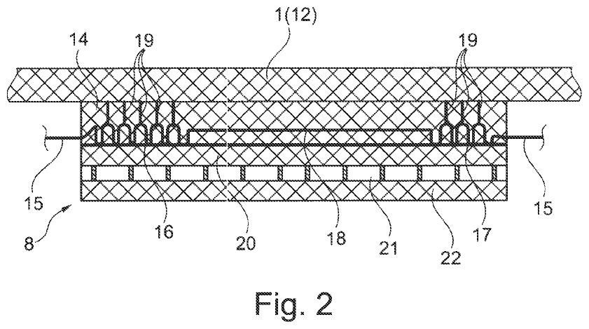

FIG. 2 shows the electrode unit 8, which is disposed on the suit 1 or on the cuff 12. The electrode unit 8 consists of a base layer 14. Two electrodes 16, 17 of an electrically conductive thread 15 are embroidered into the base layer 14. The thread 15 emerges from both sides of the base layer 14, so that an electrical circuit may be formed. Between the electrodes 16, 17, an electrically conductive connection or joint 18 is likewise formed with the thread 15. The thread 15 is embroidered on only the side of the base layer 14 turned away from the suit. The thread 15 is fixed in the base layer 14 with retaining threads 19, which are not electrically conductive. In principle, the possibility also exists that the electrodes 16, 17 are formed from two threads 15, so that two electrical circuits are present. In this case, the electrodes 16, 17 can be activated individually.

The base layer 14 consists of nonwoven fabric and is tightly fastened to the suit 1 or the cuff 12 by means of gluing, thermal pressing methods or the like. A plastic layer 20 is disposed on the side of the base layer turned away from the suit 1 or the cuff 12. A further layer of a knitted fabric, in the present case of a gauze 21, is disposed on the plastic layer 20. In addition to these layers, a further plastic layer 22 is provided.

The gauze 21 advantageously consists of an elastic material. It may consist of polyester or other materials.

The plastic films or layers 20, 22 are advantageously formed as polyurethane film. Electrically conductive particles (not illustrated) such as graphite or silver, for example, may be disposed in the plastic layers 20, 22, either in the one layer or in the other layer or in both layers. The plastic layers 20, 22 may also consist of thermoplastic material or of thermoplastic elastomers and also of conductive silicones.

The layers 14, 20, 21, 22 may be joined tightly with one another in a hot-pressing method. All described layers (12, 14, 20, 21, 22) are joined tightly with one another.

FIG. 2 shows the structure of the layers as described in accordance with the invention with the layer structure b1).

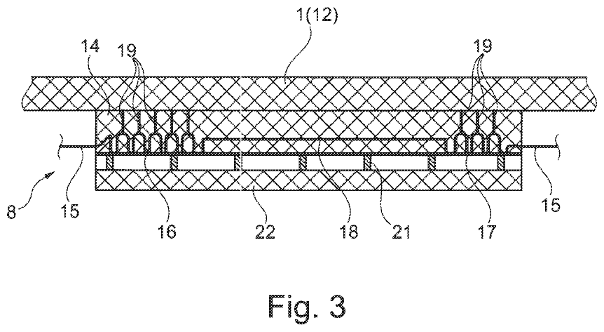

In FIG. 3, like parts as in FIG. 2 are denoted by the same reference symbols. The electrode unit 8 consists of the base layer 14 with the electrodes 16, 17 embroidered therein. The gauze 21 is disposed on the base layer 14 on the side turned away from the suit 1 or respectively the cuff 12. Furthermore, a plastic layer 22 is provided. This structure corresponds to the structure b2) embodiment of the invention. The materials are the materials that were described for FIG. 2.

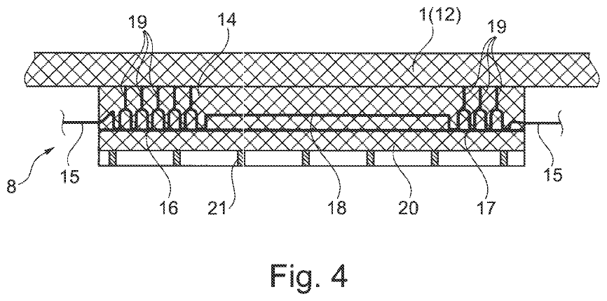

FIG. 4 shows a further possible structure. Like parts as in FIGS. 2 and 3 are denoted by the same reference symbols. The base layer 14 with the electrodes 16, 17 is disposed on the suit 1 or respectively the cuff 12. The plastic layer 20 is disposed on the side of the base layer 14 turned away from the suit 1 or the cuff 12. The gauze 21 is disposed on the plastic layer 20. This structure corresponds to the structure b3) embodiment of the invention.

The base layer 14 may consist of a nonwoven fabric, a knitted fabric, a warp-knitted fabric or the like. The electrodes 16, 17 are advantageously embroidered into the base layer 14. They may, however, also be printed, inserted, tightly glued or the like.

Due to the arrangement of the layer structure on the base layer on the side that is turned away from the suit or the cuff with at least one plastic layer 20, 22 and the gauze 21, a flexible electrode unit 8 is formed that is also durable for a long time even during long use with recurring shearing and/or twisting forces and/or stretching and/or squashing.

Although only a few embodiments of the present invention have been shown and described, it is to be understood that many changes and modifications may be made thereunto without departing from the spirit and scope of the invention.

What is claimed is:

1. A device for electrostimulation or for data acquisition by diagnostic instruments comprising:
at least one electrode unit comprising:
layer a) comprising a base layer with at least one electrode disposed in or on the base layer; and
layer b) comprising a layer having a layer structure comprising:
b1) a first plastic layer, a knitted fabric layer of knitted fabric, and a second plastic layer;
or
b2) the knitted fabric layer and the second plastic layer;
or
b3) the first plastic layer and the knitted layer;
wherein the layers a) and b) (14, 20, 21, 22) are joined with one another.

2. The device according to claim 1, wherein the at least one electrode is disposed in or on the base layer by embroidering, printing, sewing, gluing and/or inserting and fixing.

3. The device according to claim 1, wherein the base layer is a prefabricated base layer and the at least one electrode is disposed in or on the prefabricated base layer.

4. The device according to claim 1, wherein at least one of the first and second plastic layers is electrically conductive.

5. The device according to claim 1, wherein at least one of the first and second plastic layers is formed from a thermoplastic material and/or from thermoplastic elastomers and/or from conductive silicones.

6. The device according to claim 1, wherein at least one of the first and second plastic layers contains electrically conductive particles.

7. The device according to claim 6, wherein the electrically conductive particles comprise silver or graphite or silver and graphite.

8. The device according to claim 1, wherein the base layer comprises a nonwoven fabric.

9. The device according to claim 1, wherein at least one of the first and second plastic layers is a polyurethane film.

10. The device according to claim 1, wherein the knitted fabric layer is formed from polyester.

11. The device according to claim 1, wherein the knitted fabric layer contains or is formed from:
(a) gauze and/or
(b) elastic gauze and/or
(c) grid-shaped material and/or
(d) grid-shaped elastic material.

12. The device according to claim 1, wherein the base layer, the first plastic layer, the knitted fabric layer, and the second plastic layer are
(a) welded together with one another by a thermal pressing method or
(b) joined by a thermal pressing method with a primer or
(c) joined with one another by laminating or
(d) joined with one another by high-frequency welding or ultrasonic welding.

13. The device according to claim 1, further comprising an article of clothing selected from the group consisting of a whole-body suit, a partial body suit, and a cuff, wherein the base layer is disposed on the article of clothing.

14. The device according to claim 13, wherein the article of clothing comprises
(a) polyester and elastane or
(b) polyamide 6 and elastane or
(c) polyamide 6.6 and elastane or
(d) polyurethane and elastane.
